(19) United States
(12) Patent Application Publication
(10) Pub. No.: US 2019/0054260 A1
(43) Pub. Date: Feb. 21, 2019

(54) NEBULIZER DEVICES AND METHODS

(71) Applicant: Monzano Group LLC, HENDERSON, NV (US)

(72) Inventors: Chase Fahnert, Henderson, NV (US); Angelo Palmisano, Henderson, NV (US)

(21) Appl. No.: 15/679,670

(22) Filed: Aug. 17, 2017

(51) Int. Cl. A61M 15/00; A61M 11/00

(52) U.S. Cl. CPC A61M 15/0085; A61M 11/005; B05B 17/0646; A61M 15/0028; A61M 15/0065

(57) ABSTRACT

Nebulizer devices and methods are provided to facilitate a specific and controlled dosage of an atomized liquid therapy or medicine into the respiratory system of a user. The discrete nebulizer device comprises, in part, an airflow sensor connected to a controller such that inhaling through a mouthpiece ultimately activates the atomizer element to convert a liquid into an atomized liquid to maximize efficient delivery of the atomized liquid to the respiratory system of the user during inhalation. Multiple doses may be administered during sequential inhalations and exhalations without the need to remove the device from the air way opening of the user. In some embodiments, the nebulizer is programmable, hand-held, and/or disposable, for example.

### BACKGROUND

Breathing aerosolized substances for therapeutic purposes or medicinal treatments dates back at least 4,000 years. Inhalation devices were used in Assyria around 650 BC and Hippocrates described a ceramic pot-and-reed inhalation device around 400 BC. Variations on Hippocrates's device were used in the late 18th and 19th centuries. The English physician, John Mudge, was the first person to use the term “inhaler” in 1778 when he described a device for inhaling opium vapor to treat a cough.

Throughout the evolution of inhalers, drugs available for the treatment of respiratory diseases seem to have been the primary motivation behind the development of new inhalation systems including straws, pipes, atomizers, vaporizers and nebulizers. Around the turn of the 20th century, combustible powders and cigarettes containing Datura stramonium were popular for treating asthma and other lung ailments. Following the discovery of epinephrine for treating asthma, hand-bulb nebulizers were developed, as well as crude compressor nebulizers.

Nebulizers convert liquid into aerosolized particles suitable for inhalation. Nebulizers use oxygen, compressed air (e.g. jet nebulizers) or ultrasonic power (e.g. ultrasonic nebulizers) to break up a medication or therapeutic solution and deliver it in a dose of aerosolized particles to respiratory structures including the lungs.

Modern nebulizers developed for aerosolization of liquids can be traced to the mid-19th century and the advent of atomizers. Early atomizers were often referred to as “apparatus for the pulverization of liquids”. In the literature the terms “nebulizer” and “atomizer” appear to have been used synonymously during this time. Some distinguish nebulizers by the presence of a baffled spray cloud-producing device. The early atomizers were perfume atomizers and lacked a baffle system which would have created an aerosol with smaller droplets.

In the early 1930's, a compressor nebulizer (i.e. Pneumostat™) incorporated a rheostat to adjust the electrical voltage powering the compressor. The marketing of the first pressurized metered-dose inhaler for epinephrine and isoproterenol in 1956 was considered a milestone for inhalable drugs.

The ultrasonic nebulizer, which utilized a transducer made from a piezoelectric crystal, was produced in the 1960's but it was never as commercially successful as the jet nebulizer. The intermittent positive-pressure breathing machine for the delivery of bronchodilator solutions was popular in the 1980's. Reports suggested that this cumbersome device was not an improvement over nebulizers or similar inhalers. In fact, this breathing machine was associated with exacerbating asthma attacks, decreasing gas exchange, and causing lung damage, so the use of this device was discontinued. The first breath-actuated inhaler (i.e. Duo-Haler™) was manufactured by 3M Corporation in 1970. This device did not gain clinical acceptance because of its large size and the fact that it made a loud click on actuation.

Today, conventional nebulizers waste a great deal of the medication during expiration. Nebulizers are highly inefficient and may only deliver 10% of the medication to the lungs. Much of the medication is often caught on the internal apparatus and is, therefore, not available to be inhaled. The efficiency of the nebulizer may depend on the type and volume of the nebulizer chamber and the flow rate at which it is driven. Breath-assisted open vent systems may improve drug delivery but are dependent on the patient/user having adequate expiratory flow. Additionally, it has been shown that patients poorly understand the principles of nebulizer treatment and are unaware when compressors frequently malfunction (Boyter, A. C. and Carter, R.; How do patients use their nebulizer in the community? Respiratory Medicine; (2005) 99(11):1413-1417).

When using conventional constant output nebulizers as aerosol systems, the inhaled amount of drug is primarily a function of the patient's breathing pattern (i.e. inspiratory time/sum of inspiratory and expiratory times). Many new drugs have a narrow therapeutic window and are rather expensive. The delivery of these drugs requires an inhalation device which delivers a precise amount of aerosol with minimal waste and caregiver exposure. The conventional constant output nebulizer has not met these demands.

Particularly with regard to mesh nebulizers, it has been found that if the nebulizer is tilted or if the patient's head is not tipped back far enough, the fluid medication will run (under the influence of gravity) off the membrane. This can be messy and waste valuable medicine. Similarly, if the patient is bedridden or if a complex air path must be established through the nebulizer, little if any medication will be nebulized to reach the bloodstream of the patient.

Accordingly, it would be preferable to have available an improved inhalation sensitive nebulizer for use at various angles and independent of alignment with the patient's head so as to maximize the percentage of medication that reaches deep into the lung tissue and bloodstream of the patient where it can serve as effective therapy.

Clearly, many problems associated with the efficient delivery of inhaled therapeutics and medicines using a nebulizer device still exist.

Information related to attempts to address these problems can be found in U.S. Pat. Nos. 3,774,602; 4,976,259; 5,060,671; 5,165,392; 5,209,225; 5,241,954; 5,284,163; 5,293,883; 5,297,542; 5,299,565; 5,435,282; 5,331,954; 5,435,282; 5,653,223; 6,041,789; 6,443,146; 6,557,549; 6,601,581; 6,705,312; 6,729,327; 6,962,151; 7,013,894; 7,080,643; 7,819,115; 7,841,335; 8,127,772; 8,347,883; 8,353,287; 8,887,697; 9,027,548; and United States Patent Application Publication Numbers: 2008/0283049 A1; 2008/0283099 A1; 2009/0050141 A1; 2010/0020008 A1; 2011/0108025 A1; 2012/0048264 A1; 2013/0152922 A1; 2013/0192594 A1; 2013/0247910 A1; as well as International Patent Publication Numbers: PCT WO 2016019061 A1; PCT WO 2012162305 A1; PCT WO 2017007489 A1; and the following journal articles: Ari, A., Jet, Ultrasonic, and Mesh Nebulizers: An Evaluation of Nebulizers for Better Clinical Outcomes, Eurasian J. Pulmonol., (2014) 16:1-7; Ari, A., et al., Performance Comparisons of Jet and Mesh Nebulizers Using Different Interfaces in Simulated Spontaneously Breathing Adults and Children, Journal of Aerosol Medicine and Pulmonary Drug Delivery (2014) 28(0):1-9; Boyter, A. C. and Carter, R.; How do patients use their nebulizer in the community? Respiratory Medicine; (2005) 99(11):1413-1417; and Denyer, J., and Nikander, K.; The I-Neb Adaptive Aerosol Delivery (AAD) System; Medicamundi; (2010) 54(3):54-58; and Okere, N. C., Cost-Benefit Analysis of a Dosimetric Nebulizer Using Circulaire and a Traditional Vixone Nebulizer, Master's Thesis, Georgia State University, Atlanta Ga. 2011, pages 1-39, for example. Various types of nebulizer devices or associated technologies, including some embodiments of the invention, can mitigate or reduce the effect of, or even take advantage of, some or all of these potential problems. For at least the foregoing reasons, there's a legitimate need for effective and efficient methods and devices to facilitate delivery of aerosolized therapies and medications to a user including an improved inhalation sensitive nebulizer. It would be preferable that the nebulizer include a refillable liquid compartment (i.e. reservoir) or conveniently accept cartridges containing a liquid pharmaceutical. In this regard, it is also desirable that the user be able to dispense a precisely metered dose from the liquid compartment or cartridge so that the atomized therapy can be generated in a consistent droplet size and mixed with ambient atmospheric air in synchronization with the inhalation of the user. Users would like a simple (e.g. inhalation or manual switch activation), reliable, customized (e.g. treatment programmable) and discrete (e.g. hand-held and/or single use) nebulizer design. Reducing wasted medication and saving power would also be advantageous.

### BRIEF SUMMARY

These and other features, aspects, and advantages of various embodiments of the invention will become better understood with regard to the following description, appended claims, accompanying drawings and abstract.

For purposes of this disclosure, “atomize” and “nebulize” are synonymous in that a liquid substance is converted into a gas, a mist, a fog, a cloud, a smoke, an atomized liquid, aerosolized particles, or a colloid suspension of fine solid particles, all of which are capable of being inhaled, for example. Furthermore, the terms “vapor” and “aerosol” are synonymous as used herewith unless further qualified (e, . “water vapor”).

Embodiments herein provide methods and devices useful in converting a therapeutic substance or medicinal treatment from a liquid to and atomized liquid for inhalation by a user. In one aspect, the invention provides a method comprising accessing a nebulizer device, positioning a mouthpiece in contact with an airway opening of a user; and inhaling through the mouthpiece so that an air flow sensor is activated, signaling a controller, which in turn activates an atomizer element so that an atomized liquid is delivered to the airway of the user. The accessed nebulizer device comprises a housing and a controller contained in the housing. A power source is contained in the housing and connected to the controller. A liquid compartment is contained within the housing. The liquid compartment comprises an interior configured to hold a volume of liquid. An atomizer element is connected with the controller and fluidly connected with the interior. The atomizer element is configured to convert liquid into an atomized liquid during use. A mouthpiece is connected to the housing. The mouthpiece is in fluid connection with the atomizer element. An airflow sensor is connected to the controller and the housing.

In some embodiments, the airway of the user includes any anatomical region associated with a mammalian respiratory system.

In other embodiments, the atomized liquid has an aerosol particle size between about 0.3 μ and 5 μ (microns). The atomized liquid may include an aerosol particle size between about 0.5 μ and 3 μ (microns).

In still other embodiments, the mouthpiece is removably attached to the housing and the nebulizer device includes a manual switch configured to override the airflow sensor to activate the atomizer element and deliver the atomized liquid to the airway of the user. The nebulizer device may include more than one manual switch configured to be moved by the user to activate the atomizer element and deliver the atomized liquid to the airway of the user.

In another embodiment, the controller activates the atomizer element independent of inhalation. The nebulizer device may include a light configured to indicate when power is supplied upon activation of the atomizer element and the housing may include a transparent portion to view the volume of liquid remaining in the liquid compartment.

In some embodiments, the transparent portion includes visual marks approximating the volume of liquid in the liquid compartment and the velocity and/or distribution of the atomized liquid is determined by the controller. The controller may be configured to vary the frequency of the atomizer element, create back pressure in the mouthpiece, adjust a jet nozzle configured to assist with delivering the atomized liquid to the airway of the user, or any combination thereof. The controller may be configured to communicate with a remote device and the communication is a wireless communication and the remote device is a smart phone or a computer, for example. The controller may be programmed by the user or a health care professional. The programmable controller includes adjusting an activation time, a duration of activation, an interval of activation, and/or a frequency at which the atomized liquid is available for inhalation.

In other embodiments, the liquid compartment is a refillable reservoir, a standard cartridge, or a proprietary cartridge. The cartridges may be exchangeable and/or replaceable. The refillable reservoir is configured to hold a volume of liquid at different depths and the atomizer element is positioned substantially near the greatest depth of the refillable reservoir. The atomized liquid may be a therapeutic substance or a medicinal treatment.

In some embodiments, the entire nebulizer device is configured to fit the user's hand (or a health care professional's hand). At least a portion of the volume of liquid is converted into the atomized liquid by the atomizer element using ultrasonic vibration, heat, or a combination thereof. The ultrasonic vibration includes varying frequencies of the atomizer element.

In another aspect, the nebulizer device is configured to dispense a dosage of an atomized liquid. The nebulizer device comprises a housing having a distal end and a proximal end. A cartridge is configured to hold a volume of preloaded liquid. The cartridge is releasably contained within the housing. A controller is contained in the housing and configured to be programmed to dispense a dosage of the atomized liquid. A power source is also contained in the housing and connected to the controller. An atomizer element is located in the housing and connected to the controller. The atomizer element is in fluid communication with the cartridge and configured to convert at least a portion of the volume of liquid into the atomized liquid during use according to the dosage. A mouthpiece is connected to the housing and is in fluid connection with the atomizer element. An airflow sensor is connected to the controller and the housing and is configured to sense airflow from the mouthpiece. An inhalation vent on the housing is exposed to atmospheric air and is fluidly connected to the airflow sensor and the mouthpiece. An exhalation vent is on the housing and is also exposed to atmospheric air and fluidly connected to the airflow sensor and the mouthpiece. The controller is configured in such a way that, when an intake of airflow is sensed by the airflow sensor, the controller activates the atomizer element to dispense the atomized liquid and flow to the inhalation vent is opened to the mouthpiece such that the dispensed atomized substance mixes with atmospheric air via the inhalation vent to move the atomized substance into an airway of a user when the mouthpiece is substantially proximal to an opening of the airway of the user and the user is inhaling through the mouthpiece. The controller is further configured such that, when an exhalation of airflow is sensed by the airflow sensor, the exhalation is directed away from the atomizer element and out the exhalation vent.

In other embodiments, the nebulizer device is configured to dispense the dosage during a single treatment session. The dosage may be established by a health care professional or the user/patient. The opening of the airway of the user is a mouth or a nose. The dosage may be predetermined and includes any substance capable of delivery to the airway of the user via atomized liquid. The atomized liquid includes medications or therapeutic substances selected from the group consisting of anti-IGE, beta-antagonists, actinocholinergics, coricosteroids, budesonide, cromolyn sodium, ipatropium, levalbuterol, albuterol, theophylline, and tetrahydrocannabinol, for example. The dosage is based on inhalation duration, inhalation frequency, time intervals between successive inhalations, or any combination thereof. The device is a hand-held device.

In yet another aspect, a hand-held ultrasonic nebulizer device for delivering inhalable therapeutic aerosolized particles to a respiratory system of a user comprises a housing containing an inner chamber and an outer chamber. A controller is contained in the inner chamber. A power source is contained in the inner chamber and is connected to the controller. A liquid compartment is contained in the inner chamber and is comprises an interior configured to hold a volume of liquid. An atomizer element is connected with the controller and fluidly connected with the interior. A mouthpiece is connected to the housing and in fluid connection with the atomizer element. An airflow sensor is connected to the controller and the housing. The atomizer element is configured to ultrasonically change the liquid to the inhalable therapeutic aerosolized particles when the airflow sensor detects a negative pressure indicative of inhaled air. Furthermore, the outer chamber includes an inhalation vent configured to provide air from an ambient environment to flow into the mouthpiece and mix with the inhalable therapeutic aerosolized particles during inhalation. At least one exhalation vent is configured to expel exhaled air into the ambient environment.

One or more ports are located in the mouth piece. The ports are essentially holes or outlets to accommodate the ambient environment. This configuration allows airflow in a direction such that the user can successively inhale the therapeutic aerosolized particles from the atomizer element and exhale through the exhalation vent without affecting the atomizer element and without removing the nebulizer device from the opening of the respiratory system during use. This conveniently allows the user to take multiple inhalations and exhalations in succession without removing the nebulizer device from the mouth or other airway opening.

In some embodiments, the liquid compartment is a reservoir that can be refilled. The reservoir is configured to hold the volume of liquid at different depths and the atomizer element is located at the greatest depth of the refillable reservoir. The liquid compartment is configured to interchangeably accept at least one cartridge and the cartridge is filled with the volume of liquid.

### BRIEF DESCRIPTION OF THE DRAWINGS

FIG. 1A is a schematic representation depicting the formation of droplets of liquid in a gas as a result of high-amplitude surface waves.

FIG. 1B is a graphical representation showing differences in distribution by frequency over time/distance using three different frequency settings.

FIG. 2A is a cross-section bottom view of an embodiment of the nebulizer device with a liquid compartment comprising a refillable reservoir.

FIG. 2B is perspective view of the nebulizer device shown in FIG. 2A proximal to an airway opening of a user.

FIG. 2C is a cross-section side view of an embodiment of the liquid compartment of the nebulizer device shown in FIGS. 2A and 2B.

FIG. 3A is a cross-sectional view of another embodiment of the nebulizer device with a liquid compartment comprising a cartridge.

FIG. 3B is a cross-sectional view of another embodiment of the nebulizer device with a safety feature.

FIG. 4 is a cross-section bottom view of another embodiment of the nebulizer device with an airflow sensor.

FIG. 5A is a cross-section bottom view of another embodiment of the nebulizer device.

FIG. 5B is a cross-section side view of an embodiment of the nebulizer device showing air flow direction through inhalation and exhalation vents.

FIG. 5C is a cross-section bottom view of another embodiment of the nebulizer device including mouthpiece and port configurations.

FIG. 6 is a graphical representation showing differences in oxygen vs. medication over time/distance using two combinations of oxygen and medication levels.

FIG. 7A is a topographical representation showing relatively fast medication delivery with relatively high frequency and less forced oxygen over time.

FIG. 7B is a topographical representation showing relatively slower medication delivery with relatively more forced oxygen over time.

FIG. 8 depicts the remote programming and/or monitoring of the nebulizer device by a user or health care professional.

Other features of the present embodiments will be apparent from the accompanying drawings and from the detailed description that follows.

### DETAILED DESCRIPTION

Certain embodiments of the invention effectively and efficiently facilitate a specific and controlled dosage of an atomized liquid therapy or medicine into the respiratory system of a user. Multiple doses may be administered during sequential inhalations and exhalations without the need to remove the device from the air way opening of the user. In some embodiments, the nebulizer is programmable, hand-held, and/or disposable, for example.

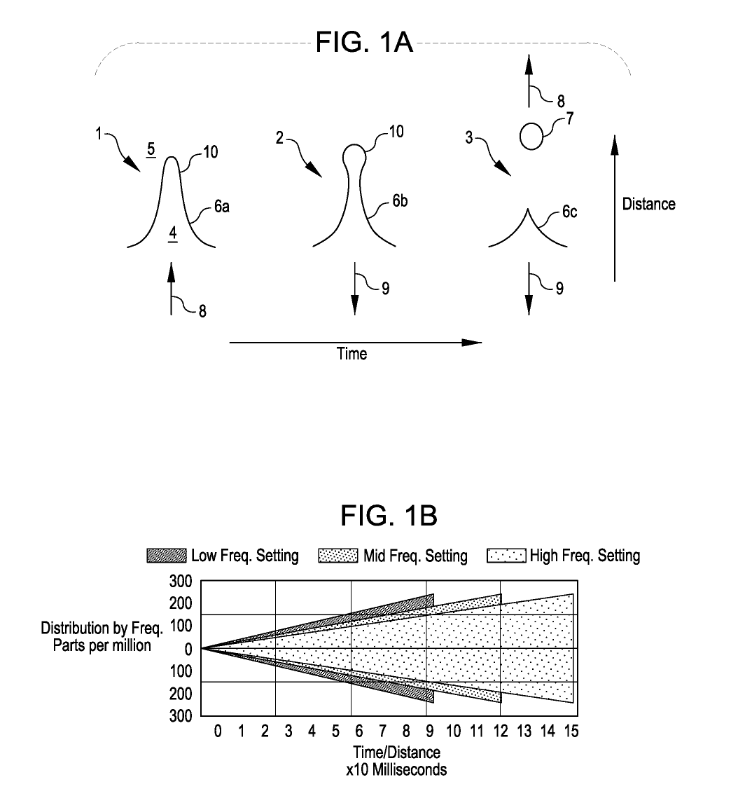

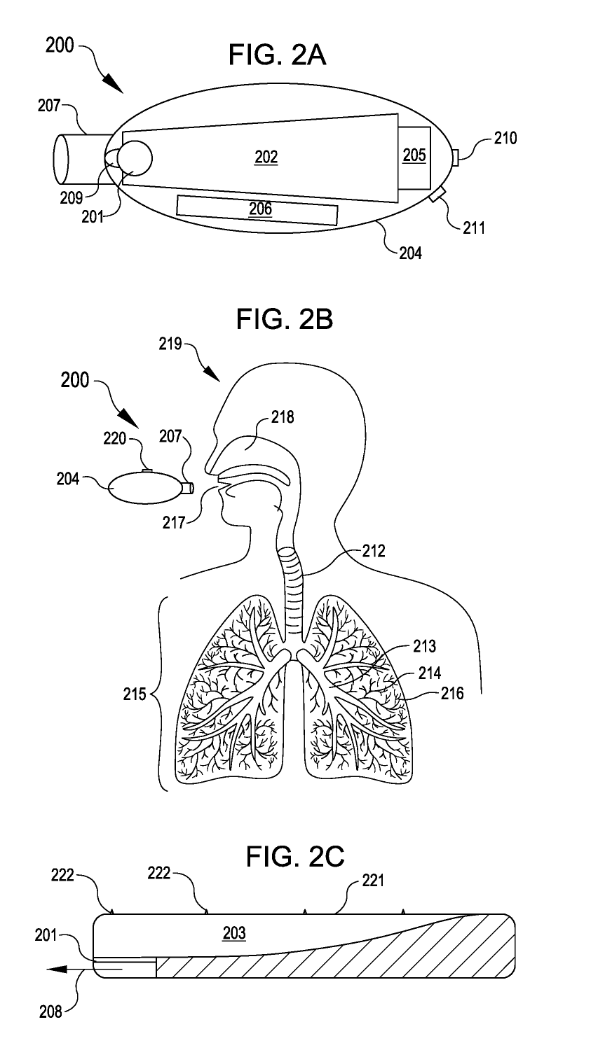

FIG. 1A depicts wave propagation through a liquid 4 using an atomizer element to apply ultrasonic vibration. An example of atomizer elements 201, 401, 301, 501 are shown in FIGS. 2A, 3A, 4 and 5A, respectively. This process causes surface waves, such as wave 6a, to be formed on any gas 5 interface in its path. The displacement amplitude of this surface wave increases as the intensity of the driving frequency is increased. Three stages 1, 2, 3 occur during one half-cycle of this surface wave 6a, 6b, 6c, respectively. One half-wavelength of the surface wave 6a is moving out into the gas 5 in the direction 8. As shown in stage 2, the direction of motion 9 is reversed but the tip 10 of the liquid column tends to continue to move into the gas 5 under its own inertia and is restrained mainly by cohesive and surface tension forces. At a high enough displacement amplitude, these forces are overcome and a droplet 7 of the liquid 4 breaks free and enters the gas 5 as shown in stage 3. Similar processes may occur during the next half-cycle of the surface wave which may result in a bubble of gas being formed within the liquid. It has been determined experimentally that the diameters of the liquid droplets produced by this mechanism are about 0.34 times the wavelength of the surface wave providing that only small quantities of the liquid are being atomized.

The frequency of the atomizer element may be varied or adjusted to increase or decrease the ultrasonic vibration. This variation may be pre-programed or self-configured by a user or health care professional. FIG. 1B, shows differences in distribution by frequency over time/distance using three different frequency settings (i.e., low, medium and high).

The frequency of the ultrasonic nebulizer determines the particle size of the aerosol. The particle size generally falls within the range of 0.5 μ to 3.0 μ (microns). The amplitude or strength of these sound waves determines the output of the nebulizer, which is usually in the range of about 0 to 3 ml/minute and 0 to 6 ml/minute, for example. The ultrasonic nebulizer may also incorporate a fan unit to move the aerosol to the patient. This fan helps cool the device. It is anticipated that the gas flow generated by the fan falls in the range of between 21 and 35 liters/minute.

In addition to ultrasonic vibration, a liquid may be atomized by the atomizer element by heating a volume of the liquid. This variation may also be pre-programed or self-configured by a user or health care professional. Ultrasonic vibration and evaporation via heating the liquid may be used in combination to atomize a volume of liquid as well.

FIG. 2A is a cross-section bottom view of an embodiment of the nebulizer device 200 with a liquid compartment 202 comprising a refillable reservoir 203 (FIG. 2C). The nebulizer device comprises a housing 204 and a controller 205 contained in the housing. Typically, a microchip and the controller 205 are mounted on a single board. In practice, their functions can blend into a single chip with control functions on the chip. The controller may be a standard control (i.e., a device or mechanism used to regulate or guide the operation of a machine, apparatus, or system), a microcomputer, or any other device that can execute computer-executable instructions, such as program modules. Generally, program modules include routines, programs, objects, components, data structures and the like that perform particular tasks or implement particular abstract data types. A programmer of ordinary skill in the art can program or configure the controller to perform the functions described herein. As an example, the controller 205 determines the distribution of the atomized liquid. A power source 206 is contained in the housing 204 and connected to the controller 205. The power source may be single-use or rechargeable battery, for example. The liquid compartment 202 contained within the housing 204 has an interior configured to hold a volume of liquid. FIG. 2C is a side view of this feature without the mouthpiece attached. The refillable reservoir 203 of the liquid compartment 202 is shallow towards the anterior (i.e., front) of the device and has the greatest depth over the atomizer element 201. In this manner, gravity feeds the atomizer element the liquid medicine or therapeutic agent. The reservoir 203 may include a transparent material 221 such as clear plastic and include convenient measurement markers 222 approximating the volume of liquid in the liquid compartment. These markers 222 may be expressed in milliliters (ml), for example. A lid may be attached with a hinge or other similar mechanism to close access to the liquid compartment 202.

An atomizer element 201 is connected with the controller 205 and fluidly connected with the interior and configured to convert liquid into an atomized liquid during use. The atomizer element is positioned horizontally and assists to force air down and out towards the mouthpiece 207 in direction 208. FIG. 2A shows the mouthpiece 207 is connected to the housing 204 and in fluid connection with the atomizer element 201. An airflow sensor 209 is connected to the controller 205 and the housing 204.

During use, as shown in FIG. 2B, the mouthpiece 207 is placed in contact with an airway opening (e.g., mouth 217 or nose 218) of a user 219. When the user 219 inhales through the mouthpiece 207, the air flow sensor 209 is activated, signaling the controller 205, which in turn activates the atomizer element 201 so that the atomized liquid is delivered to the airway (e.g., trachea 212, bronchus 213, bronchioles 214, lungs 215, alveoli 216, etc.) of the user 219. The controller 205 may also activate the atomizer element 201 independent of inhalation sensed by the air flow sensor 209 based on a programmed time or a manual switch override, for example.

The nebulizer device 200 may include a manual switch 210 configured to override the airflow sensor 209 to activate the atomizer element 210 and deliver the atomized liquid to the airway of the user 219. More than one manual switch 210, 211 may be configured to be moved by the user to activate the atomizer element 201 and deliver the atomized liquid to the airway of the user. Regardless of how the atomizer element 201 is activated, illumination of a light 220 indicates when power is supplied upon activation of the atomizer element.

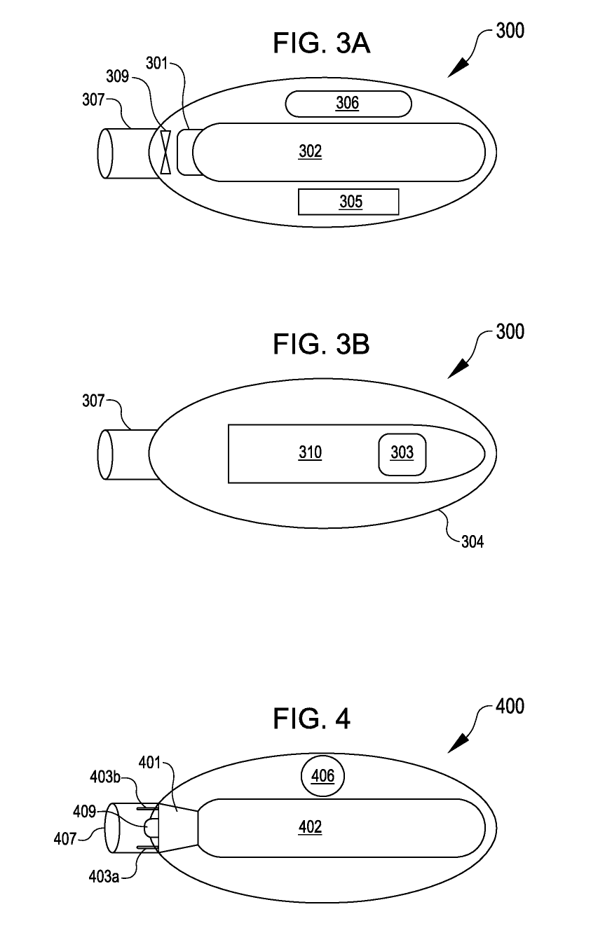

FIG. 3A illustrates another embodiment of the nebulizer device 300 that is designed for pre-filled therapies including currently existing medications. The pre-filled medication may be in the form of a cartridge or capsule 302 which is placed in the liquid compartment area and pierced by a needle or similar device to open the cartridge when the user is ready to begin treatment. The needle is connected to internal conduit that sends the flow of liquid from the cartridge 302 to the atomizer element 301. The cartridge 302 may be pressurized to facilitate delivery of medication independent of the angle at which the device is held during use (i.e., inhalation and/or exhalation).

The atomizer element 301 has the ability to encapsulate without piercing. This may be accomplished via multiple controls including any combination of: 1) actuation; 2) program schedule; 3) biometrics; and 4) remote application. As shown in FIG. 3B, safety features 303, located within a liquid-proof compartment 310, may allow combinations of these methods. Safety features may include a button or combination of buttons that can be manually pressed by the user to lockdown the nebulizer device to prevent others from using the device. In this manner, misuse or abuse of the medication by others is eliminated. The manual lockdown feature may be particularly advantageous in a disposable or single use version of the nebulizer device to prevent the device from inadvertent activation when stored in a pocket or purse, for example. The refillable (i.e., non-disposable) nebulizer may have a lockdown device including fingerprint activation or require a password to activate. The activation may be integrated into the nebulizer or activation may be wirelessly transmitted to the nebulizer via a smart phone application, for example. Additionally, a proximity locator may be used to access the device. Other similar controls to limit access to the device are contemplated and are familiar to those of skill in the art such that the examples described herewith, or any combination thereof, should not limit other such safety mechanisms or features commonly known in the art.

FIG. 4 is a cross-section bottom view of another embodiment of the nebulizer device 400 with an airflow sensor 409 surrounded by a conically-shaped atomizer element 401. A power source 406 and a cartridge 402 are also included. The cartridge may be a proprietary or standard cartridge. The cartridges may also be exchanged or replaced from the liquid compartment. The mouthpiece 407 may be removable to facilitate cleaning, disposal, or replacement. If the mouthpiece 407 is removable, it may be configured with rails 403a, 403b on the right and left sides to allow the mouthpiece to slide off the housing 404, for example.

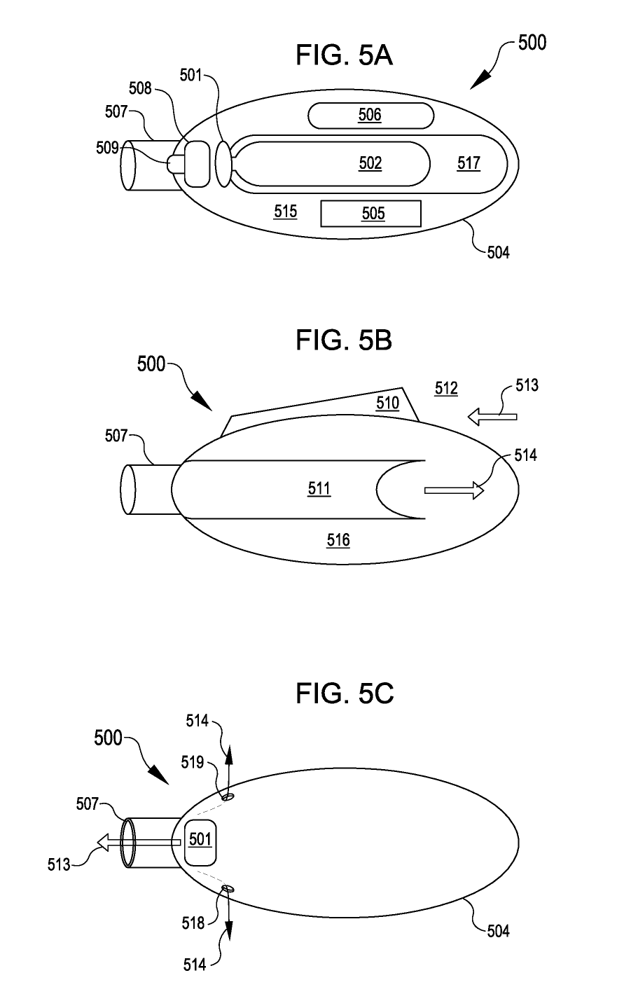

FIG. 5A is a cross-section bottom view of another embodiment of the nebulizer device 500 configured to dispense a dosage of an atomized liquid. The nebulizer device 500 comprises a housing 504 and a cartridge 502 configured to hold a volume of preloaded liquid. The cartridge 502 is releasably contained within the housing 504. A controller 505 is contained in the housing 504 and configured to be programmed to dispense a dosage of the atomized liquid. A power source 506 is also contained in the housing 504 and connected to the controller 505. The atomizer element 501 is located in the housing 504 and connected to the controller 505 and in fluid communication with the cartridge 502. The atomizer element 501 converts at least a portion of the volume of liquid into the atomized liquid during use according to the dosage. The mouthpiece 507 is connected to the housing 504 and in fluid connection with the atomizer element 501. An airflow sensor 509 is connected to the controller 505 and the housing 504 and configured to sense airflow from the mouthpiece 507.

As shown in FIG. 5B, an inhalation vent 510 on the housing 504 is exposed to atmospheric air 512 and fluidly connected to the airflow sensor and the mouthpiece 507. The direction 513 of air 512 drawn into the nebulizer device 500 during an inhalation by a user is shown in FIG. 5B. An exhalation vent 511 is also located on the housing 504 and exposed to atmospheric air 512. The exhalation vent 511 is fluidly connected to the airflow sensor and the mouthpiece 507. The controller 505 is configured such that, when an intake of airflow 513 is sensed by the airflow sensor 509, the controller 505 activates the atomizer element 501 to dispense the atomized liquid and flow to the inhalation vent 510 is opened to the mouthpiece 507 such that the dispensed atomized substance mixes with atmospheric air 512 via the inhalation vent 510 to move the atomized liquid into an airway of a user when the mouthpiece 507 is substantially proximal to an opening of the airway of the user and the user is inhaling through the mouthpiece 507. Refer also to FIGS. 2B and 8. The controller 505 is further configured so that when an exhalation of airflow is sensed by the airflow sensor 509, the exhalation is directed away from the atomizer element and out the exhalation vent 511 in direction 514.

In another embodiment shown in FIGS. 5A and 5B, a hand-held ultrasonic nebulizer device 500 delivers inhalable therapeutic aerosolized particles (of atomized liquid) to a respiratory system of a user. The nebulizer includes a housing 504 containing an inner chamber 515 and an outer chamber 516. A controller 505 and power source 506 are connected to each other and contained in the inner chamber 515. A liquid compartment 517 is contained in the inner chamber 515 and comprises an interior configured to hold a volume of liquid. An atomizer element 501 is connected with the controller 505 and fluidly connected with the interior. A mouthpiece 507 is connected to the housing 504 and in fluid connection with the atomizer element 501. An airflow sensor 509 is connected to the controller 505 and the housing 504. In this embodiment, the atomizer element 501 is configured to ultrasonically change the liquid to the inhalable therapeutic aerosolized particles when the airflow sensor 509 detects a negative pressure indicative of inhaled air from direction 513.

The outer chamber 516 includes an inhalation vent 510 configured to provide air 512 from an ambient environment to flow into the mouthpiece 507 and mix with the inhalable therapeutic aerosolized particles during inhalation. At least one exhalation vent 511 is configured to expel exhaled air into the ambient environment in the direction 514. One or more ports 518, 519 are located in the mouth piece 507. Port 518 (FIG. 5C) ties into exhalation vent 511 (FIG. 5B). The ports 518, 519 are essentially holes or outlets to accommodate exhalation into the ambient environment. This configuration allows airflow in a direction such that the user can successively inhale in direction 513 while drawing the therapeutic aerosolized particles from the atomizer element 501 and air 512 from the inhalation vent 510 and exhale through the exhalation vent 511 without affecting the atomizer element and without removing the nebulizer device from the opening of the respiratory system (e.g., mouth) during use. This conveniently allows the user to take multiple inhalations and exhalations in succession without removing the nebulizer device from the mouth or other airway opening. Since the atomizer element is only activated during inhalation, energy is also conserved.

The nebulizer device 500 may be configured to dispense the dosage established by a health care professional or a user during a single treatment session. As shown in FIG. 2b, the opening of the airway of the user 219 is a mouth 217 or a nose 218, although it is conceivable the nebulizer device would also effectively deliver atomized liquid in accordance with embodiments of the present invention via stoma or even as an auxiliary attachment to a medical ventilator.

The dosage of atomized liquid may be predetermined and could include medications or therapies such as anti-IGE, beta-antagonists, actinocholinergics, bronchodilators, cortiosteroids, budesonide, cromolyn sodium, ipatropium, levalbuterol, albuterol, theophylline, and tetrahydrocannabinol, for example. The dosage may be based on inhalation duration, inhalation frequency, time intervals between successive inhalations, or any combination of these parameters.

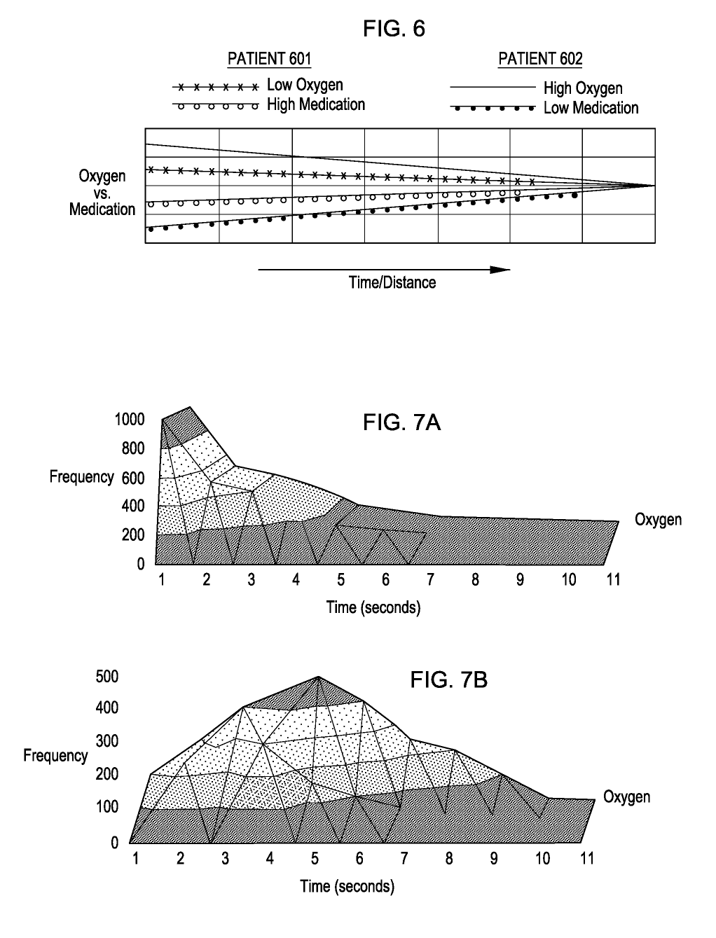

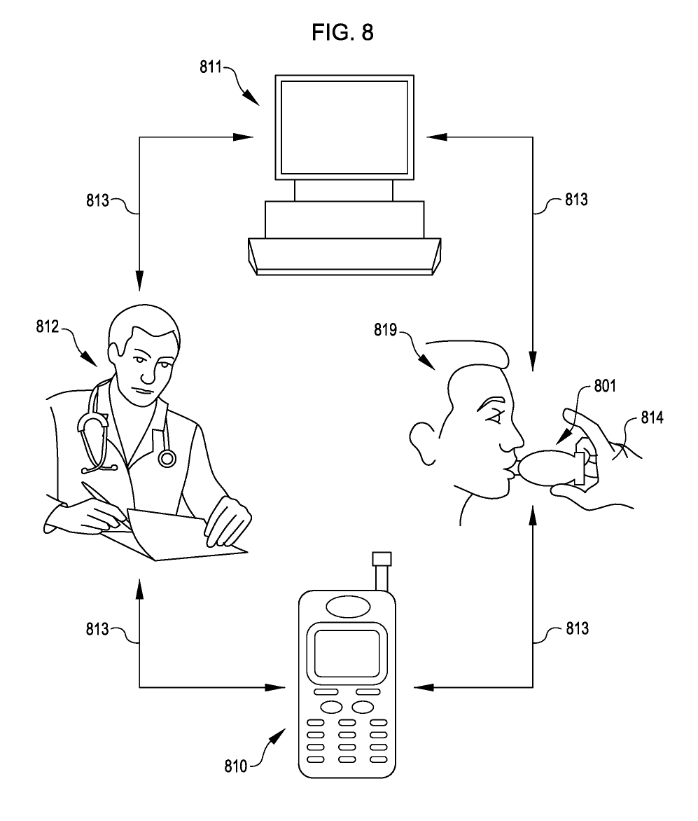

As shown in FIG. 6, patient 601 could potentially require a stronger mist jet to force the atomized medication past the restriction and all the way into the lungs with a full dose of medication (i.e., high medication) being delivered relatively fast. Patient 601 could be a child with acute restricting airways caused by an asthma attack, for example. FIG. 7A is a topographical representation showing relatively fast medication delivery with relatively high frequency and less forced oxygen.

Conversely, patient 602 may require a longer, slower treatment with more air (i.e., high oxygen) and less mist (i.e., low medication) to allow the medication to disperse in a longer, slower absorption. In this example, patient 602 may be an elderly person that exhibits a slow, mild restriction. Breathing atomized liquids (e.g., water) may be almost as effective as inhaling the medication itself for patient 602. This is similar in effect to using a humidifier in a controlled fashion. FIG. 7B is a topographical representation showing relatively slower medication delivery with relatively more forced oxygen.

A variance in medication concentration can be obtained by adjusting the air-to-mist ratio. By creating a relative longer exposure to the air/mist combination, different therapeutic results can be obtained based off exposure time and other factors. By regulating the rate of the atomization and potentially the force at which atomized particles are delivered, a higher medication absorption rate may be obtained. Of course, various other combinations may be used depending on specific circumstances including using a high oxygen and high medication setting or a low oxygen and low medication setting, for example.

As previously described with respect to FIG. 1B and further now to FIG. 8, the variable velocity/distribution of the atomized liquid is controlled by the frequency which the nebulizer 810 operates. The frequency is controlled by the technology in the nebulizer; e.g., by instructions maintained in the controller. The frequency can be set at a single setting, or can be adjusted, for example via the connecting technology 813. In such an embodiment, the connecting technology can be used to send requested frequency to the controller from a smart phone 801, computer 811, or another input device. Once the data is received, the controller will adjust the frequency of the atomizer element.

The nebulizer 801 may include a user profile which enforces/reinforces various functions of the nebulizer. These functions may include, for example: 1) changing the air-to-mist ratio delivered over time so as to vary the amount of medication ultimately delivered to the user 819; 2) restricting the activation of the nebulizer such that the nebulizer device can only be used during designated times; accepting only specifically designated cartridges into the liquid compartment; and/or administering a partial dosage.

The entirety of the nebulizer 801 is configured to fit in a hand 814 of a user 819 (or health care professional 812) and include control features that allow a user 819 or health care professional 812 to configure the nebulizer 801 to match a prescription including, but not limited to: multiple uses from a single cartridge, air-to-mist ratio, time enforcing rules, frequency/velocity, etc.

The nebulizer device 801 may be preconfigured to atomize medication based on specific personalized criteria. Examples of such criteria may include the user's lung capacity, airway size, amount of air that is passed per inhalation/exhalation, and the velocity of which the air is passed in combination with patient age, gender, weight, and existing medical conditions, for example.

Alternatively or additionally, the nebulizer 801 may be self-configurable using a smart phone 810 or computer 811 and connected 813 via Wi-Fi or Bluetooth™, for example. In this manner, the controller, housed inside the nebulizer device 801, communicates with a remote device (i.e. phone 810 or computer 813). Preferably, the communication is wireless. The nebulizer 801 efficiently delivers an atomized medication to the user 819 based on user-selected criteria or a prescription from a health care professional 812.

It is further contemplated that the nebulizer may send data to an application and, depending on criteria already selected by the user 819 or a health care professional 812 (e.g., gender, age, etc.), the application will then use the data to arrive at an atomization and velocity protocol specifically designed to fulfill the personal requirements of the user 819 or the prescription dictated by a health care professional 812.

The previously described embodiments of the subject invention have many advantages, including an improved inhalation sensitive nebulizer, with a refillable liquid compartment (i.e. reservoir) or exchangeable pre-filled cartridges containing a liquid pharmaceutical or therapy. In this regard, the user is able to easily dispense a precisely metered dose from the reservoir or cartridge so that the atomized liquid can be generated in a consistent droplet size and dispensed in synchronization with the inhalation of the user. A simple, reliable, customizable, and discrete nebulizer device is disclosed herewith that mitigates wasted medication and saves power.

Other variations are within the spirit of the present invention. Thus, while the invention is susceptible to various modifications and alternative constructions, certain illustrated embodiments thereof are shown in the drawings and have been described above in detail. The location of the various components (i.e., drawing elements) of the nebulizer device may be changed. For example, compare power source 206 in FIG. 2A with power source 306 in FIG. 3A. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

Although embodiments of the invention have been described in considerable detail with reference to certain preferred versions thereof, other embodiments are possible. Therefore, the spirit and scope of the appended claims should not be limited to the descriptions of the embodiments above.

What is claimed is:

1. A method comprising:
accessing a nebulizer device comprising:
a housing;
a controller contained in the housing;
a power source contained in the housing and connected to the controller;
a liquid compartment contained within the housing and comprising an interior configured to hold a volume of liquid;
an atomizer element connected with the controller and fluidly connected with the interior and configured to convert liquid into an atomized liquid during use;
a mouthpiece connected to the housing and in fluid connection with the atomizer element; and
an airflow sensor connected to the controller and the housing;
positioning the mouthpiece in contact with an airway opening of a user; and
inhaling through the mouthpiece so that the air flow sensor is activated, signaling the controller, which in turn activates the atomizer element so that the atomized liquid is delivered to the airway of the user.

2. The method of claim 1, wherein the airway of the user includes any anatomical region associated with a mammalian respiratory system.

3. The method of claim 1, wherein the atomized liquid has an aerosol particle size between about 0.3 and 5 microns.

4. The method of claim 3, wherein the atomized liquid has an aerosol particle size between about 0.5 and 3 microns.

5. The method of claim 1, wherein the mouthpiece is removably attached to the housing.

6. The method of claim 1, wherein the nebulizer device includes a manual switch configured to override the airflow sensor to activate the atomizer element and deliver the atomized liquid to the airway of the user.

7. The method of claim 6, wherein the nebulizer device includes more than one manual switch configured to be moved by the user to activate the atomizer element and deliver the atomized liquid to the airway of the user.

8. The method of claim 1, wherein the controller activates the atomizer element independent of inhalation.

9. The method of claim 1, wherein the nebulizer device includes a light configured to indicate when power is supplied upon activation of the atomizer element.

10. The method of claim 1, wherein the housing includes a transparent portion to view the volume of liquid remaining in the liquid compartment.

11. The method of claim 10, wherein the transparent portion includes visual marks approximating the volume of liquid in the liquid compartment.

12. The method of claim 1, wherein the velocity and/or distribution of the atomized liquid is determined by the controller.

13. The method of claim 1, wherein the controller is configured to vary the frequency of the atomizer element, create back pressure in the mouthpiece, adjust a jet nozzle configured to assist with delivering the atomized liquid to the airway of the user, or any combination thereof.

14. The method of claim 1, wherein the controller is configured to communicate with a remote device.

15. The method of claim 14, wherein the communication is a wireless communication and the remote device is a smart phone or a computer.

16. The method of claim 1, wherein the controller is programmed by the user or a health care professional.

17. The method of claim 1, wherein the programmable controller includes adjusting an activation time, a duration of activation, an interval of activation, and/or a frequency at which the atomized liquid is available for inhalation.

18. The method of claim 1, wherein the liquid compartment is a refillable reservoir, a standard cartridge, or a proprietary cartridge.

19. The method of claim 18, wherein the cartridges are exchangeable and/or replaceable.

20. The method of claim 18, wherein the refillable reservoir is configured to hold a volume of liquid at different depths and the atomizer element is positioned substantially near the greatest depth of the refillable reservoir.

21. The method of claim 1, wherein the atomized liquid is a therapeutic substance or a medicinal treatment.

22. The method of claim 1, wherein the nebulizer device is configured to fit in a hand of the user.

23. The method of claim 1, wherein at least a portion of the volume of liquid is converted into the atomized liquid by the atomizer element using ultrasonic vibration, heat, or a combination thereof.

24. The method of claim 23, wherein the ultrasonic vibration includes varying frequencies of the atomizer element.

25. A nebulizer device configured to dispense a dosage of an atomized liquid, the nebulizer device comprising:
a) a housing having a distal end and a proximal end;
b) a cartridge configured to hold a volume of preloaded liquid, the cartridge releasably contained within the housing;
c) a controller contained in the housing and configured to be programmed to dispense a dosage of the atomized liquid;
d) a power source contained in the housing and connected to the controller;
e) an atomizer element located in the housing, the atomizer element connected to the controller and in fluid communication with the cartridge and configured to convert at least a portion of the volume of liquid into the atomized liquid during use according to the dosage;
f) a mouthpiece connected to the housing and in fluid connection with the atomizer element;
g) an airflow sensor connected to the controller and the housing and configured to sense airflow from the mouthpiece;
h) an inhalation vent on the housing and exposed to atmospheric air and fluidly connected to the airflow sensor and the mouthpiece; and
i) an exhalation vent on the housing and exposed to atmospheric air and fluidly connected to the airflow sensor and the mouthpiece;
wherein the controller is configured such that, when an intake of airflow is sensed by the airflow sensor, the controller activates the atomizer element to dispense the atomized liquid and flow to the inhalation vent is opened to the mouthpiece such that the dispensed atomized substance mixes with atmospheric air via the inhalation vent to move the atomized substance into an airway of a user when the mouthpiece is substantially proximal to an opening of the airway of the user and the user is inhaling through the mouthpiece; and
wherein the controller is further configured such that, when an exhalation of airflow is sensed by the airflow sensor, the exhalation is directed away from the atomizer element and out the exhalation vent.

26. The nebulizer device of claim 25, wherein the device is configured to dispense the dosage during a single treatment session.

27. The nebulizer device of claim 25, wherein the dosage is established by a health care professional or the user.

28. The nebulizer device of claim 25, wherein the opening of the airway of the user is a mouth or a nose.

29. The nebulizer device of claim 25, wherein the dosage is predetermined and includes any substance capable of delivery to the airway of the user via atomized liquid.

30. The nebulizer device of claim 25, wherein the atomized liquid includes medications or therapeutic substances selected from the group consisting of anti-IGE, beta-antagonists, actinocholinergics, bronchodilators, corticosteroids, budesonide, cromolyn sodium, ipatropium, levalbuterol, albuterol, theophylline, and tetrahydrocannabinol.

31. The nebulizer device of claim 25, wherein the dosage is based on inhalation duration, inhalation frequency, time intervals between successive inhalations, or any combination thereof

32. The nebulizer device of claim 25, wherein the device is a hand-held device.

33. A hand-held ultrasonic nebulizer device for delivering inhalable therapeutic aerosolized particles to a respiratory system of a user, the device comprising:
a housing containing an inner chamber and an outer chamber;
a controller contained in the inner chamber;
a power source contained in the inner chamber and connected to the controller;
a liquid compartment contained in the inner chamber and comprising an interior configured to hold a volume of liquid;
an atomizer element connected with the controller and fluidly connected with the interior;
a mouthpiece connected to the housing and in fluid connection with the atomizer element; and
an airflow sensor connected to the controller and the housing;
wherein the atomizer element is configured to ultrasonically change the liquid to the inhalable therapeutic aerosolized particles when the airflow sensor detects a negative pressure indicative of inhaled air; and wherein the outer chamber includes:
1) an inhalation vent configured to provide air from an ambient environment to flow into the mouthpiece and mix with the inhalable therapeutic aerosolized particles during inhalation; and
2) at least one exhalation vent configured to expel exhaled air into the ambient environment.

34. The device of claim 33, wherein the liquid compartment is a refillable reservoir configured to hold the volume of liquid at different depths and the atomizer element is located at the greatest depth of the refillable reservoir.

35. The device of claim 33, wherein the liquid compartment is configured to interchangeably accept at least one cartridge, the cartridge filled with the volume of liquid.
